(19) United States
(12) Patent Application Publication
(10) Pub. No.: US 2023/0062473 A1
(43) Pub. Date: Mar. 2, 2023

(54) BONE MARROW ASPIRATION AND BIOPSY DEVICE

(71) Applicant: PURE MARROW INC., Barrington, RI (US)

(72) Inventors: Paul Tessier, Killingworth, CT (US); James Elliott Brown, Barrington, RI (US)

(21) Appl. No.: 17/794,863

(22) PCT Filed: Jan. 25, 2021
(86) PCT No.: PCT/US2021/014881

§ 371 Date: Jul. 22, 2022

Related U.S. Application Data
(60) Provisional application No. 62/965,673, filed on Jan. 24, 2020.

(51) Int. Cl. A61B 10/02

(52) U.S. Cl. CPC A61B 10/025; A61B 10/0283; A61B 10/0275; A61B 2010/0258

(57) ABSTRACT

A biopsy and aspiration device that can include an outer cannula that has a plurality of apertures formed in an outer surface, a biopsy stylet insertable into the outer cannula having a beveled tip portion and slits formed in the main body, and an aspiration stylet also separately insertable into the outer cannula having a plurality of apertures formed in the main body and an optional sealing element associated therewith. One or more of the apertures of the aspiration stylet can be aligned with one or more of the apertures of the outer cannula.

### RELATED APPLICATION

The present invention claims priority to U.S. provisional patent application Ser. No. 62/965,673, entitled BONE MARROW ASPIRATION AND BIOPSY DEVICE, filed on Jan. 24, 2020, the contents of which are herein incorporated by reference.

### BACKGROUND OF THE INVENTION

The present invention is related to biopsy devices, and more specifically is related to bone marrow biopsy devices.

Bone marrow aspiration and biopsy (BMAB) devices are typically used to diagnose and monitor blood cancers and are the principal source of tissue sample for blood cancer research. Bone marrow aspiration (BMA) is conventionally performed by puncturing the cortex of the pelvic bone with a standard biopsy needle and applying rapid, high vacuum extraction via syringe to the marrow cavity. The primary goal of the BMA is to dislodge a spicule (e.g., a bit of marrow parenchyma the size of a grain of sand) so that it can be prepared on a microscope slide and interpreted by a pathologist. Empirical studies now show that the sensitivity of BMA for detecting malignancy is suboptimal.

In an attempt to obtain an adequate sample, clinicians frequently aspirate a significant volume of marrow which can be counterproductive. High volume aspiration typically draws in venous blood rather than the desired marrow and spicules (i.e., hemodilution). The reliability of BMA worsens with increasing volumes taken from the same location due to worsening hemodilution and small vacuum pressures, which fail to dislodge spicules; therefore, it is highly operator or technique dependent. It remains a significant limitation despite optimized checklists and systematic techniques underscoring the limits of current sampling tools.

Hemodilution confounds the ratio of mature to immature (blast) cells, the gold standard, for example, for the classification of Myelodysplastic Syndrome (MDS) and Acute Myelogenous Leukemia (AML) and is a major factor in reducing diagnostic sensitivity. For example, if an aspirate containing 2 ml of marrow and 20% blasts (e.g., acute leukemia) is diluted by 8 ml of peripheral blood containing no blasts, the blast count can decrease to 4% (within normal range). Hence, hemodilution can result in highly variable blast percentages.

Some cancer diseases are more affected by a poor aspirate than others. For example, in MDS, blast percentages of greater than 25% is indicative of progression to leukemia and therefore this count highly influences decisions regarding patient treatment and prognosis. When flow cytometry is used to control for hemodilution in blast counts, classification changes often. Given such a low percentage, a small amount of hemodilution can significantly affect the disease detection threshold. Methods to control for hemodilution post-sampling are usually not practical or not applicable in most situations.

The effect of hemodilution is also evident in studies of children with acute lymphoblastic leukemia (ALL), where a hemodilute specimen changed risk stratification, decreasing blast percentage 54%, and in 4/14 patients decreased the blast percentage below the >0.1% threshold for minimal residual disease. Lymphoid neoplasms are also affected by poor aspirates. In one study, BMA detected bone marrow infiltration by the aggressive subtype of lymphoma 10% of the time when the core biopsy was negative, despite only a 40% sensitivity of BMA in that series. Collective of all disorders, if the BMA fails and the BMB is not diagnostic, repeat BMAB is performed. A prospective evaluation of 838 BMAB samples found a non-diagnostic rate of up to 8.5%, resulting in an estimated 76,500 unnecessary procedures each year.

Further, a retrospective study of 5000 aspirates at a specialized referral center found a combined frequency of suboptimal or failed aspirates 27% of the time. BMA is therefore combined with a bone marrow biopsy (BMB) to improve sensitivity. With most currently available sampling tools, BMB requires a second bone puncture and additional advanced laboratory analysis also dependent on BMA quality (flow cytometry and cytogenetics) and yet half of the 27% limited aspirates remain suboptimal. A second bone puncture also increases procedure time and patient discomfort.

BMA is also a source of moderate to severe pain in patients. Patient pain remains under-treated because physicians underestimate the patient's pain level and are often not equipped to offer sedation or other forms of pain management or mitigation techniques. In a prospective randomized trial of a low-level of sedation such as oral pain medication (e.g., sublingual fentanyl) prior to BMAB surprisingly did not lessen the patients' perception of pain. Pain during aspiration is likely caused by negative pressure from the syringe suction needed to extract the marrow as pain is correlated to pressure changes in bone and is experienced more acutely in cancer patients where the baseline bone pressure is already abnormal. Pain during BMA deters patients' willingness to participate in clinical trials requiring additional BMA, undermining the field's ability to advance cancer treatment.

Thus, conventional bone marrow aspiration and biopsy devices have at least four significant issues that need improvement: 1) aspicular samples arising from the random distribution of spicule-containing hematopoietic marrow combined with a small sampling area; 2) hemodilution arising when samples are diluted with the venous blood that bathes the marrow parenchyma, which preferentially flows during aspiration due to low viscosity; 3) the inability to take a core biopsy and an aspirate sample at the same core sample site without the aspirate ruining the core biopsy; and 4) patient pain.

The issue of hemodilution is also a problem when performing bone marrow aspiration to harvest stem cells for regenerative medicine treatments. It has been shown that treatment results are highly correlated with stem cell dose.

The focus of innovation in this field has been on trying to reduce hemodilution. To avoid hemodilution, it is necessary to draw samples from multiple locations within the marrow. Utilizing multiple bone puncture sites is undesirable and is only used for marrow harvesting for life saving transplant procedures. Thus, the focus has been on trying to access different regions of marrow from a single bone puncture. Attempted solutions include multiple side holes in a marrow sampling needle and techniques to change the location of the sampling hole or aperture within the bone.

Placing multiple side holes in a marrow sampling needle has the issue of flow primarily occurring from the hole with the least resistance, and therefore sampling does not universally occur at each side location. To overcome this limitation, techniques and devices have been developed to move the location of the sampling holes or apertures within the bone.

One such device accomplishes repositioning by having a flexible drill shaft that the user advances. The flexible drill shaft slides along the inner table of the hard, outer bone obtaining a large aspiration field. However, this method depends on a normal bone density to deflect the needle. Many patients who need bone marrow harvesting/sampling are older adults who have low bone density and therefore there is a risk the shaft could penetrate the bone causing severe injury to surrounding organs and internal bleeding.

Another device accomplishes the multiple depth sampling technique by pulling back the needle which exposes a side hole or aperture to marrow at different depths. This solution work by anchoring a threaded outer handle onto the skin of the patient which connects to a threaded handle connected to the active aspirate needle into the bone. By turning the outer handle, the threads make sensitive changes in penetration depth of the needle that make for multiple areas of sampling. However, in patients with a prominent amount of subcutaneous fat, the movement of the needle in the bone is limited by the compressibility of the fat, the fat being compressed with the turning of the needle rather than creating movement of the needle in the bone.

In additional to the issues outlined above, users have found operation of these devices complicated. Therefore, there is a need to improve the art.

### SUMMARY OF THE INVENTION

The present invention is directed to a tissue penetrating device that includes an outer cannula and different inner cannula components that can be employed to perform different functions. The outer cannula can include a main body that has one or more apertures or holes formed therein, and the apertures can be arranged in any selected pattern, such as for example in a spiral or linear pattern. According to one embodiment, the inner cannula component can be a biopsy stylet for removing a portion of tissue from the patient. The biopsy stylet of the present invention can have a main body that includes opposed slits formed therein and a beveled tip portion. The slits terminate in a keyhole end region. The beveled tip portion facilitates the cutting of the tissue during the biopsy procedure. Moreover, it is beveled inwardly so that the diameter of the core of tissue that is cut corresponds generally to the outer diameter rather than the inner diameter. Therefore, the tissue core is minimally or slightly compressed as it enters the opening o the stylet, which aids in capturing and retaining the tissue therein. The slits form a pair of opposed, flexible grasper portions that allow the end portion of the biopsy to resiliently expand outwardly to be close to or at the inner diameter of the outer cannula so as to retrieve a tissue sample as large as possible and to provide gentle compression to maximize recovery of the tissue.

The inner cannula component can also be an aspiration stylet according to the teachings of the present invention. The aspiration stylet can have a main body that has one or more apertures or holes formed therein. The apertures can be arranged in any selected pattern, and can be arranged in a pattern that is the same as or different than the apertures formed in the outer cannula. The aspiration stylet can also include one or more optional sealing components mounted on the outer surface thereof or about or within one or more of the apertures. The sealing component forms a fluid tight seal between the outer cannula and the aspiration stylet. Alternatively, the main body of the aspiration stylet can be configured to form a sealing portion, such as by forming a bulge or expanded portion in the main body that is intended to make intimate facing contact with the inner surface of the outer cannula to form a fluid tight seal. The aspiration stylet can be moved either axially, rotationally or both, such that one or more of the apertures can be aligned with one or more apertures of the outer cannula. Any selected one of the apertures of the outer cannula can be aligned with any selected one of the apertures of the aspiration stylet. According to one exemplary practice, the apertures of the outer cannula can be aligned with one of the apertures of the aspiration stylet one at a time in sequence. This aperture alignment allows tissue, such as bone marrow, to be aspirated into the central or inner region of the aspiration stylet. The aspiration stylet can also have multiple inner lumens that are isolated from each other along the central opening. This configuration can allow separate tissue sampling from the distal end of the device. Alternatively, it can allow fluid from a fluid source to pass through one of the lumens and be introduced to the surgical site. Further, the other or remaining lumen can be employed to aspirate and retain the tissue. The introduction of a fluid, such as saline, to the surgical site reduces pain to the patient by reducing the pressure differential at the tissue extraction site. Further, the fluid introduction helps reduce hemodilution.

The present invention is directed to a tissue penetrating device, comprising an outer cannula having a main body having a tip portion formed at a distal end and a plurality of apertures formed at selected locations along an axial length thereof, the main body also forming a central opening that extends along the axial length; a hub portion coupled to a proximal end of the outer cannula; a biopsy stylet having a main body forming a central opening and having a tip portion formed at a distal end thereof having a beveled end portion, wherein the main body has a pair of opposed slits formed in the distal end and axially extending from the tip portion a selected length along the main body, wherein the slits formed in the biopsy stylet form at the distal end of the main body a pair of flexible opposed stylet halves; and a handle portion coupled to a proximal end of the biopsy stylet. Further, the biopsy stylet has an outer diameter smaller than an inner diameter of the outer cannula so as to seat within the central opening of the outer cannula and the handle has a selected portion that is sized and configured to seat within the hub portion.

The tip portion of the outer cannula can include a plurality of prongs. Further, the biopsy stylet is movable relative to the outer cannula, and the tip portion has a plurality of notches formed therein.

According to another aspect, the present invention is directed to a tissue penetrating device, comprising an outer cannula having a main body having a tip portion formed at a distal end and a plurality of apertures formed at selected locations along an axial length thereof, the main body also forming a central opening that extends along the axial length; a hub portion coupled to a proximal end of the outer cannula; an aspiration stylet having a main body having a tip portion formed at a distal end and a plurality of apertures formed at selected locations along an axial length thereof, the main body forming a central opening; and a handle portion coupled to a proximal end of the aspiration stylet. The aspiration stylet has an outer diameter smaller than an inner diameter of the outer cannula so as to seat within the central opening of the outer cannula and the handle has a selected portion that is sized and configured to seat within the hub portion.

The tip portion of the outer cannula comprises a plurality of prongs and the main body of the aspiration stylet has a central opening that includes a plurality of lumens that are fluidly isolated from each other. With regard to the lumens, a first lumen of the plurality of lumens has an open end at the tip portion of the main body of the aspiration stylet and a second lumen of the plurality of lumens has a closed end at the tip portion. Further, the first lumen is configured and disposed to be in fluid communication with a fluid source for delivering a fluid through the open end of the tip portion, and the second lumen is configured and disposed to be in fluid communication with a vacuum source for aspirating tissue through one or more of the apertures of the outer cannula and one or more apertures of the aspiration stylet.

According to another aspect, the plurality of apertures formed in the main body of the aspiration stylet are arranged such that when the main body of the aspiration stylet is moved one or more of the plurality of apertures is disposed in registration with one or more of the plurality of apertures of the outer cannula. The aspiration stylet further includes one or more sealing elements disposed on an outer surface of the main body. Alternatively, the main body of the aspiration stylet can include an expansion portion for forming a seal between an outer surface of the aspiration stylet and an inner surface of the outer cannula.

The present invention is also directed to a method for aspirating tissue, comprising inserting an outer cannula within tissue of a patient at a selected location, wherein the outer cannula has a main body having a plurality of apertures formed at selected locations along an axial length thereof, the main body also forming a central opening that extends along the axial length thereof; inserting a biopsy stylet within the central opening of the outer cannula and into the tissue of the patient so as to cut and retain a portion of the tissue, wherein the biopsy stylet is axially and rotationally movable relative to the outer cannula; removing the biopsy stylet from the central opening of the outer cannula to thus remove the portion of the tissue; inserting an aspiration stylet into the central opening of the outer cannula and into the tissue of the patient, wherein the aspiration stylet has a main body having a plurality of apertures formed at selected locations along an axial length thereof; and manipulating the aspiration stylet such that one or more of the apertures of the aspiration stylet align with one or more of the apertures of the outer cannula.

Further, the biopsy stylet can be inserted into the central opening of the outer cannula, and the method can further include inserting together the outer cannula and the biopsy stylet into the tissue of the patient, wherein the tissue is cut during the insertion of the biopsy stylet. The aspiration stylet can further include one or more sealing elements mounted on an outer surface thereof, the method further comprising positioning the aspiration stylet such that the one or more sealing elements seals one or more of the plurality of apertures of the outer cannula.

The method can also include according to another aspect manipulating the aspiration stylet such that one or more of the plurality of apertures of the outer cannula are aligned with one or more of the plurality of apertures of the aspiration stylet in sequence starting from the distal most aperture of the outer cannula. According to another aspect, the aspiration stylet can be manipulated such that all of the plurality of apertures of the outer cannula are aligned with all of the plurality of apertures of the aspiration stylet.

The present invention can also be directed to a method for aspirating tissue, comprising providing an outer cannula having a main body having a plurality of apertures formed at selected locations along an axial length thereof, the main body also forming a central opening that extends along the axial length thereof, the outer cannula being configured to be inserted into tissue of a patient; providing an aspiration stylet sized and configured to be insertable within the central opening of the outer cannula and into the tissue of the patient, wherein the aspiration stylet has a main body having a plurality of apertures formed at selected locations along an axial length thereof; and manipulating the aspiration stylet when inserted into the central opening such that one or more of the apertures of the aspiration stylet align with one or more of the apertures of the outer cannula.

The present invention can also be directed to a method for providing a biopsy and aspiration of tissue, comprising providing an outer cannula having a main body having a plurality of apertures formed at selected locations along an axial length thereof, the main body also forming a central opening that extends along the axial length thereof, the outer cannula being configured to be inserted into tissue of a patient; providing a biopsy stylet sized and configured to be insertable within the central opening of the outer cannula and into the tissue of the patient so as to cut and retain a portion of the tissue when inserted therein, wherein the biopsy stylet is axially and rotationally movable relative to the outer cannula which can remain stationary; providing an aspiration stylet sized and configured to be insertable within the central opening of the outer cannula and into the tissue of the patient, wherein the aspiration stylet has a main body having a plurality of apertures formed at selected locations along an axial length thereof; and manipulating the aspiration stylet when inserted into the central opening such that one or more of the apertures of the aspiration stylet align with one or more of the apertures of the outer cannula.

The present invention can also be directed to a tissue penetrating kit. The kit can include an outer cannula having a hub portion coupled to a proximal end and a tip portion formed at a distal end, the outer cannula having a main body having a plurality of apertures formed at selected locations along an axial length thereof, the main body also forming a central opening that extends along the axial length; a biopsy stylet having a first handle portion coupled to a proximal end and a tip portion formed at a distal end, the biopsy stylet having a main body having a pair of opposed slits formed in the distal end and axially extending from the tip portion a selected length along the main body, wherein the tip portion has a beveled end portion and a plurality of notches formed therein, wherein the slits formed in the biopsy stylet form at the distal end of the main body a pair of flexible opposed stylet halves, and wherein the biopsy stylet has an outer diameter smaller than an inner diameter of the outer cannula; and an aspiration stylet having a second handle portion coupled to a proximal end and a tip portion formed at a distal end, wherein an outer diameter of the aspiration stylet is smaller than an inner diameter of the outer cannula, and wherein the aspiration stylet has a main body having a tip portion formed at a distal end and a plurality of apertures formed at selected locations along an axial length thereof.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a general schematic representation of the tissue penetrating device according to the teachings of the present invention.

FIG. 2 is a schematic configuration of one embodiment of the tissue penetrating device of the present invention showing an inner biopsy stylet disposed within an outer cannula.

FIG. 3 is a cross-sectional view of the tissue penetrating device with an inner dual lumen aspiration stylet according to the teachings of the present invention.

FIG. 4 is a perspective view of the outer cannula component and associated hub portion of the tissue penetrating device of the present invention.

FIG. 5 is a partial cross-sectional view of the tip portion or distal end of the outer cannula portion of the tissue penetrating device showing the tip portion to penetrate tissue, a hollow inner portion, and multiple linearly aligned aperture, according to the teachings of the present invention.

FIG. 6 is a schematic cross-sectional view of an inner cannula component, such as a biopsy stylet, of the tissue penetrating device according to the teachings of the present invention.

FIG. 7 is a partial exploded view of the tip portion of the biopsy stylet functioning as the inner cannula component of the tissue penetrating device according to the teachings of the present invention.

FIG. 8 is a partial cross-sectional view of the tip portion of the biopsy stylet according to the teachings of the present invention.

FIG. 9 is a partial cross-section view of the cannula assembly implementing the outer cannula and the inner biopsy stylet according to the teachings of the present invention.

FIG. 10A is a perspective view of the aspiration stylet according to the teachings of the present invention.

FIG. 10B is a partial perspective view of the tip portion of the aspiration stylet with optional sealing elements according to the teachings of the present invention.

FIG. 11 is a perspective view partly in phantom showing the inner lumen structure of the aspiration stylet according to the teachings of the present invention.

FIG. 12 is a partial cross-sectional view of the aspiration stylet according to the teachings of the present invention.

FIG. 13 is a perspective view partly in phantom showing the cannula assembly employing the outer cannula and the aspiration stylet, and illustrating aligned apertures, according to the teachings of the present invention.

FIG. 14 is a perspective view partly in phantom showing the cannula assembly employing the outer cannula and the aspiration stylet, and illustrating one or more closed apertures in the outer cannula, according to the teachings of the present invention.

### DETAILED DESCRIPTION

The present invention is directed to a tissue penetrating device, such as for example a bone marrow aspiration and biopsy device, for allowing multiple-depth tissue sampling for acquiring or extracting a sample of tissue, such as a bone marrow sample, from a patient at a surgical site. The tissue penetrating device of the present invention allows a medical professional, at a single site or location, to biopsy tissue and then to separately aspirate tissue, such as bone marrow, using separate or different inner cannula components. As used herein, the term “tissue” is intended to mean any assemblage or ensemble of cells in a body, such as a human or animal body. Examples of the types of tissue contemplated to be biopsied or aspirated by the present invention can include bone, organs, connective, muscular, nervous, epithelial, and skin, including the dermis and epidermis.

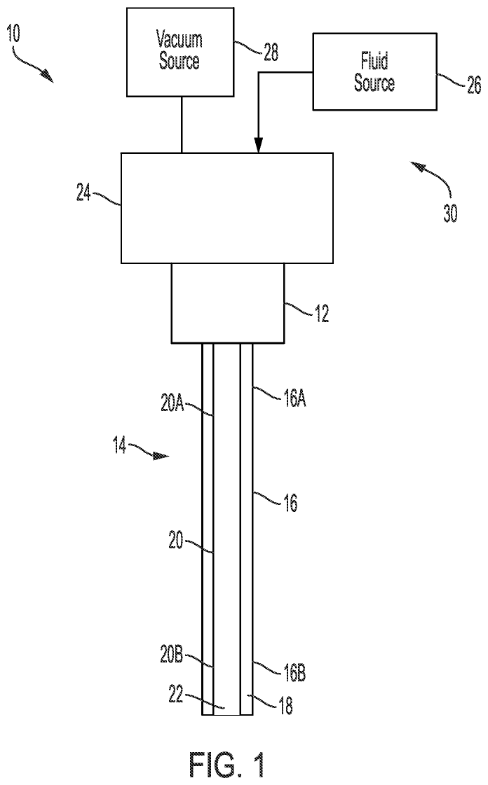

The tissue penetrating device 10 of the present is shown schematically, in a general manner, in FIG. 1. The illustrated device 10 includes a hub portion 12 that secures or has attached thereto an outer cannula 16. The outer cannular can preferably be a tissue penetrating cannula as is known in the art. Specifically, a proximal end 16A of the outer cannular 16 can be secured to the hub portion 12 and an opposed distal end 16B can be configured for penetrating the tissue of the patient. The outer cannula 16 can also include a central opening 18. The hub portion 12 can be coupled to a handle 24 that can have an inner cannula component 20 attached or secured thereto. That is, a proximal end 20A of the inner cannular component 20 is secured or attached to the handle, and the handle 24 is intended to mechanically interface with the hub portion 12 such that the inner cannula component 20 is inserted into the central opening 18 of the outer cannula. The inner cannula component 20 has an inner opening 22. The inner cannular component 20 when attached to the handle 24 is configured to be axially and rotationally movable within the opening 18 of the outer cannula 16. The outer cannula 16 and the inner cannula component 20 form a cannula assembly 14. The hub portion 12 can thus function as a connecting or docking portion for receiving the handle portions of separate inner cannula components. The inner cannula component 20 can be any selected device that has an inner or central opening that is sized and configured for axial and rotational travel within the outer cannula. Examples of suitable types of devices having an inner opening include needles, medicament administering devices, aspiration devices, biopsy devices, catheters, trocars, stylets, and the like. The tissue penetrating device 10 of the present invention is generically and schematically illustrated in FIG. 1 since many different types of combinations of cannulas 16, 20 can be employed as part of the device 10 of the present invention. According to one embodiment, the tissue penetrating device 10 can be a bone marrow aspiration and biopsy device or assembly. Those of ordinary skill in the art will readily recognize that the hub 12 and the handle 24 can have any selected shape or configuration and can have any selected size suitable for the intended surgical purpose of the device.

The tissue penetrating device 10 can be coupled to a fluid source 26 and to a vacuum source 28 to form a tissue penetrating system 30. The fluid source 26 can be fluidly coupled to the handle 24 and to an inner lumen or opening of either the outer cannula 16 or the inner cannula component 20. According to one example, the fluid source 26 is fluidly coupled with the inner or central opening of the inner cannula component to supply a fluid therethrough to the surgical site. The fluid can be any suitable biologically compatible fluid, such as saline or a saline solution. The saline solution can be used to reduce or eliminate the pressure differential at the surgical site when the device is used to remove tissue via aspiration. The illustrated vacuum source 28 is fluidly coupled to the device 10 via the handle 24 or the hub 12 so as to apply a vacuum pressure to the surgical site so as to aspirate tissue through the outer cannula 16, through the inner cannula component 20, or through both.

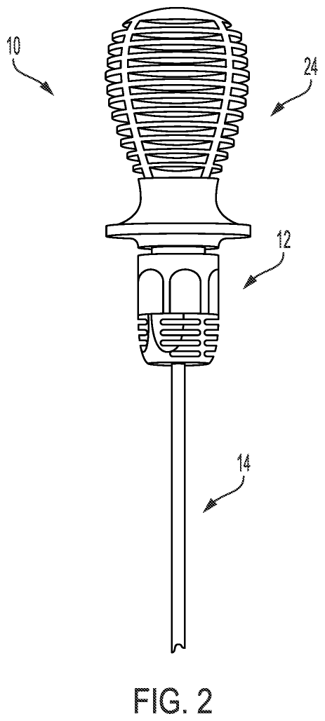

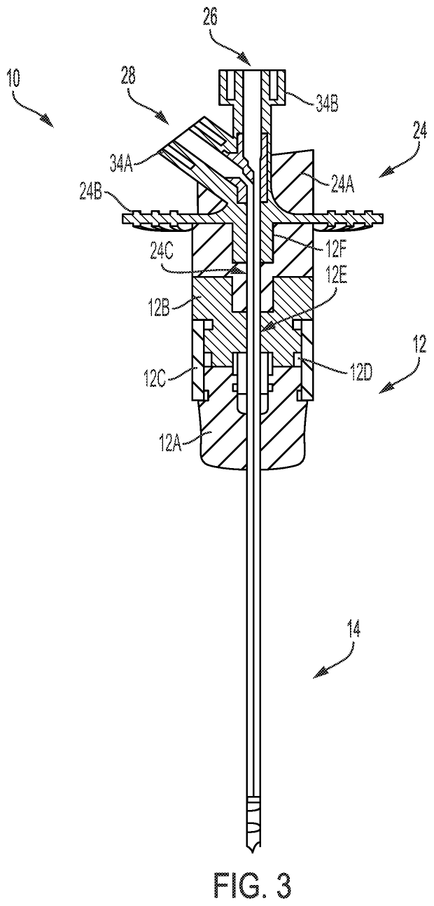

FIGS. 2 and 3 illustrate one embodiment of the tissue penetrating device 10 of the present invention. As shown, the cannula assembly 14 is coupled to a hub portion 12 and to the handle 24. The hub 12 can have any selected configuration and can comprise any selected number of linked components. According to one illustrative and exemplary embodiment, the hub 12 can include hub portions 12A and 12B that are linked together via a collar 12C. The hub 12 can also include a plurality of sealing elements 12D that form fluid tight seals along adjacent component surfaces. The seals help prevent leakage of fluids applied to the device 12 or aspirated through the device, as well as air from entering the device. The hub portions 12A, 12B when aligned and assembled also form a central opening 12E for accommodating and retaining the outer cannula 16. Further, the illustrated handle 24 can be mechanically coupled to the hub via any conventional securing or fastening technique, generically illustrated as opening 12F, and can include a main body 24A that includes an optional skirt 24B that is configured for allowing the medical professional to easily engage therewith so as to easily and readily manipulate the device 10. The handle 24 also includes a central opening 24C such that when the handle is mounted to the hub 12, the opening is aligned with the opening 12E to form a continuous fluid conduit through the device 10. According to one embodiment, either or both of the outer cannula 16 and the inner cannula component 20 can seat within the opening 24C. The device 10 also has separate intake ports 34A and 34B for receiving fluid from the fluid source 26 and pressure from the vacuum source 28, respectively. The fluid from the fluid source 26 can be introduced to one of the outer cannula 16 and the inner cannula component 20 and the vacuum source 28 can also be coupled to one of the outer cannula 16 and the inner cannula component 20.

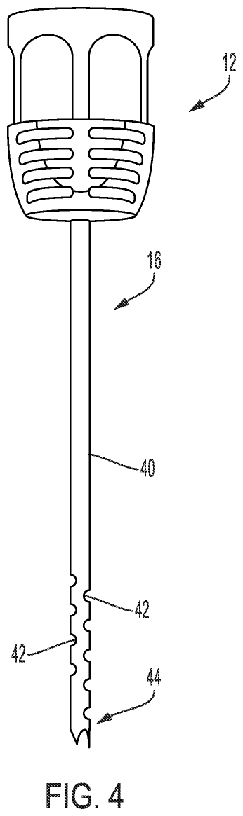

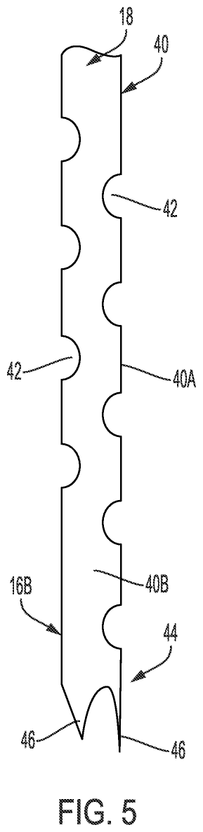

As shown in FIGS. 4 and 5, the outer cannula 16 can have a main body 40 that has an outer surface 40A and an opposed inner surface 40B. The main body forms the opening 18 that functions as an inner lumen or central opening. Those of ordinary skill will readily recognize that the outer cannula 16 can have multiple lumens arranged in any selected configuration. The main body 40 can also include one or more apertures or holes 42 formed in the main body 40 that can be arranged in any selected pattern, and are preferably separated both axially and circumferentially about the outer surface 40A. According to one embodiment, the apertures are arranged in a spiral pattern at different axial and circumferential positions about the circumference of the outer cannula 16. The apertures can be sized and shaped so as to allow the aspiration of tissue therethrough and to optimize strength given the expected axial and torsional loads of entering tissue and bone. The shape of the apertures 42 can be utilized to maximize the number of apertures placed in the outer surface 40A, thereby maximizing the tissue spaces, such as bone marrow spaces, that are sampled. The main body 40 also includes a tip portion 44 that can have a plurality of sharpened prongs 46 formed thereon for penetrating the tissue and bone of the patient. Once inserted into the tissue and/or bone of the patient, it is not necessary to remove, move, or rotate the outer cannula.

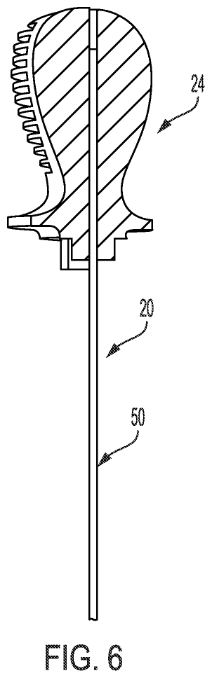

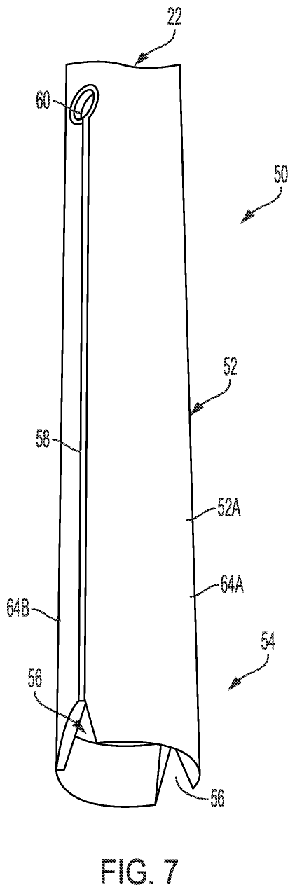

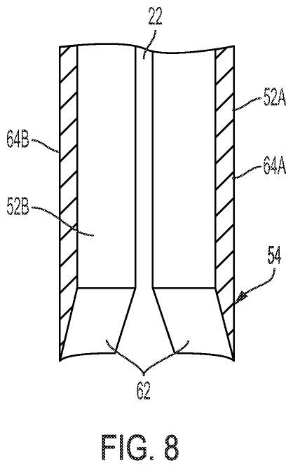

FIGS. 6-9 illustrate one example of a type of inner cannula component 20 that can be inserted into the opening 18 of the outer cannula 16 and form part of the tissue penetrating device 10 of the present invention. The inner cannula component 20 can be, for example, and according to one embodiment, a tissue removing stylet, such as for example a biopsy stylet 50, for removing tissue from the patient. The biopsy stylet 50 can be axially positioned within the opening 18 of the outer cannula 16 at any selected axial and rotational position. The illustrated biopsy stylet 50 has a main body 52 that has an outer surface 52A and an opposed inner surface 52B forming the central opening 22. The biopsy stylet 50 also includes a tip portion 54 that includes one or more notches 56 formed therein. According to one embodiment, a pair of opposed notches 56 can be formed in the tip portion 54. A pair of axially extending slits 58 are formed at opposed positions in the main body 52 to form stylet halves 64A, 64B. The slits 58 extend a selected axial distance along the main body of the stylet from the notches 56 to a selected end region or keyhole 60. The keyhole 60 forms a pivot or flex point for the body halves 64A, 64B that are formed by the slits in the main body. Further, the illustrated tip portion 54 has a beveled surface 62 formed along the inner surface 52B. The beveled surface 62 formed in the tip portion 54 creates a sharpened tip that is capable of cutting and separating a portion of tissue during a tissue removal or biopsy procedure. For example, according to one embodiment, the beveled tip can be angled inwardly such that the diameter at the terminal of the tip portion and the entry point of the lumen has a diameter larger than the diameter of the central opening 22. Specifically, the beveled surface 62 is angled inwardly so that the diameter of the core of tissue that is cut corresponds generally to the outer diameter rather than the inner diameter. Therefore, the tissue core is minimally compressed as it enters the central opening, which aids in capturing the tissue therein. The pivoting movement of the stylet halves 64A, 64B creates a flexible region that can allow the halves and thus the inner diameter at the tip portion 54 to expand and flex outwardly up to the inner diameter of the outer cannula 16, thus functioning as a spring-loaded mechanism. The flexing or pivoting movement of the stylet halves 64A, 64B during the cutting procedure ensures that the cut tissue is as large as possible. Moreover, the flexible halves can also serve to compress and retain the tissue sample once cut within the opening 22. Thus, the combination of the internally beveled tip portion 62 and halves 64A, 64B allows for improved compression and maximum diameter of the tissue core. Additional tissue catching features formed in or on the inner surface of the stylet can also be employed so as to aid in capturing different types of tissue.

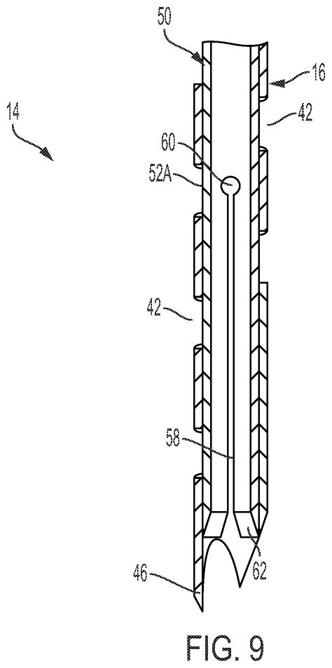

Further, as shown in FIG. 9, when the biopsy stylet 50 is inserted within the opening 18 of the outer cannula 16, the outer surface 52A of the main body 52 of the biopsy stylet 50 occludes the apertures 42 of the outer cannula. The occluded apertures 42 thus do not remove any tissue when inserted within the patient.

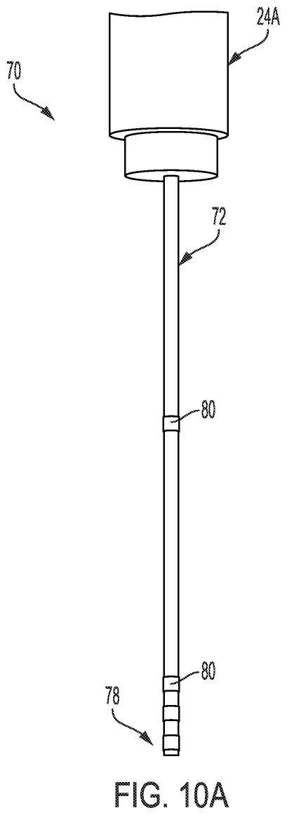

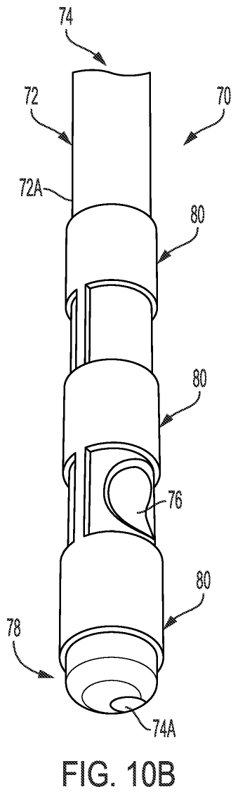

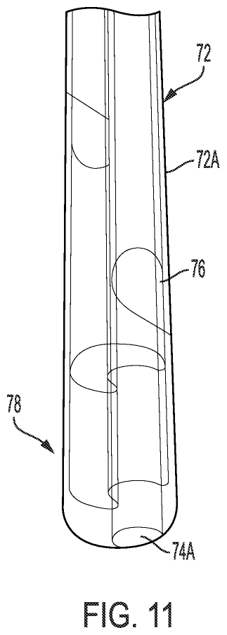

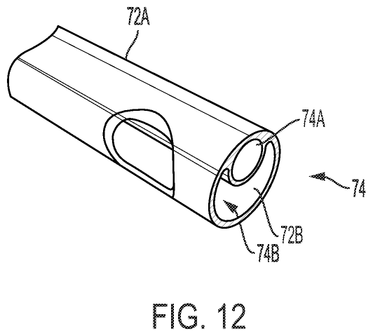

The biopsy stylet 50 can be removed from the outer cannula 16 by the user and then another device, such as a stylet or cannula, can be inserted therein. One example of a stylet that can be inserted in the outer cannula is the aspiration stylet 70, shown or example in FIGS. 10-14. The aspiration stylet 70 is configured for aspirating or withdrawing a volume of tissue from a surgical site, as is known in the art. The aspiration stylet 70 is axially and rotationally movable in the opening 18 such that the surgeon can selectively position the stylet 70 within the outer cannula 16. The illustrated aspiration stylet 70 has a main body 72 having an outer surface 72A and an opposed inner surface 72B that is coupled, and preferably secured, to a handle 24A. The handle 24A can be the same as or different than the handle 24. The inner surface 72B can form a single opening or lumen or can be configured to form multiple lumens. For example, the opening 74 can be configured to provide a first lumen 74A and a second lumen 74B, as shown for example in FIG. 12. Those of ordinary skill in the art will readily recognize that the opening 74 can be configured to include any selected number of lumens. The first lumen 72A can terminate at an opening at a tip portion 78 for allowing a fluid to flow through the length of the main body 72 and to be expelled to the surgical site. Alternatively, the lumen 72A can be used to aspirate a fluid under vacuum therethrough. According to one embodiment, the second lumen or opening 74B can be closed at the distal end so as to prevent a fluid entering or being expelled from the lumen at the tip portion 78.

The illustrated aspiration stylet 70 can also include one or more apertures 76 formed in the main body 72. The apertures 76 can be arranged in any selected pattern, and are preferably separated both axially and circumferentially about the outer surface 72A. According to one embodiment, the apertures 76 are arranged in a spiral pattern at different axial and circumferential positions about the circumference of the aspiration stylet 70. The pattern of apertures can be the same as or different than the pattern of apertures 42 formed in the outer cannula 16. The apertures 76 can be sized and shaped so as to allow the aspiration of tissue therethrough. The shape of the apertures 76 can also be utilized to maximize the number of apertures placed in the outer surface 72A, thereby maximizing the marrow spaces that are sampled. The apertures 76 of the aspiration stylet 70 can be positioned to be in registration or aligned with the apertures 42 of the outer cannula 16. Further, the aspiration stylet 70 and the outer cannula 16 can be placed at an appropriate depth within the patient that allows tissue, such as bone marrow, to be aspirated through any one or all of the apertures. When so positioned, the vacuum source 28 can apply a vacuum pressure to the lumen 74B so as to aspirate a portion of the tissue adjacent the open apertures.

The aspiration stylet 70 can also employ or include one or more optional sealing elements 80 that can be mounted on or to the main body 72 thereof. According to one embodiment, the sealing elements 80 can be mounted on the outer surface 72A of the main body 72 so as to form a fluid seal between the outer surface 72A of the aspiration stylet 70 and the inner surface 40B of the outer cannula 16. Further, the aspiration stylet 70 can be rotated or axially moved within the opening 18 so as to position the sealing element 80 in alignment or registration with one or more of the apertures 42 of the outer cannula 16 so as to occlude the aperture and form a fluid tight seal. The illustrated stylet can employ any selected number or types of seals and they can be formed of any suitable biologically compatible material, such as for example rubber or polyether block amide (PEBAX) of various durometers. The sealing element 80 can have any selected shape or size, and can be positioned on or in the aspiration stylet 70 at any selected position. For example, the sealing element 80 can be formed in or about the apertures 76 formed on the aspiration stylet 70 to form, for example, a face seal. According to one practice, the aspiration stylet 70 need not employ sealing elements that are separate and distinct, but rather can have a sealing feature integrally formed in the main body 72 of the aspiration stylet 70. For example, the main body can include an expanded region or feature that is sized and configured to for a fluid tight seal with the inner surface 40B of the outer cannula 16. Examples of suitable expansion features includes a bulge, a flange, a skirt, and the like. According to another optional embodiment, the aspiration stylet can include an active or dynamic feature that allows the user to selectively retract into an expansion feature a portion of the main body.

The tissue penetrating device 10 of the present invention can be employed by a medical professional during a surgical procedure. The device 10 includes a first medical device component that comprises the hub 12 and the outer cannula 16 secured thereto. The hub 12 functions as a quick connect receiving or docking portion for selectively receiving different types of inner cannula components and associated handle that are configured to perform different functions. Prior to positioning the outer cannula 16 within the patient at a surgical site by the medical professional, the site can be appropriately numbed or anesthetized. Once anesthetized, the medical professional inserts a trocar into the central opening of the outer cannula, and the trocar can be used to help insert the outer cannula into the patient. Once the outer cannula is positioned in the patient, the hub portion 12 attached to the outer cannula 16 can be used to receive different types of inner cannula components. According to one example, the tissue penetrating device 10 can be employed to retrieve a tissue sample, such as bone marrow. As such, the outer cannula 16 can be inserted into the patient such that the prongs 46 of the tip portion 42 cut through the skin, tissue, fat, and bone of the patient, such that the outer cannula is positioned and anchored in the bone or bone marrow at the surgical site.

If the medical professional desires to obtain a biopsy of the patient, then the professional can insert the biopsy stylet 50 into the central opening 18 of the outer cannula 16. In the current example, prior to retrieving the bone marrow, the medical professional may wish to biopsy the bone. As such, the outer cannula can be positioned in the bone in the surgical site as noted herein, and the biopsy stylet 50 can be introduced into the opening 18 of the outer cannula 16. The biopsy stylet 50 can be positioned by the medical professional at any selected axial location and rotational position within the outer cannula 16. The beveled edge 62 of the tip portion 54 of the biopsy stylet 50 functions as a cutting tool as it enters the bone of the patient under force or pressure by the medical professional. The slits 58 formed in the main body of the stylet 50 form stylet halves 64A, 64B that are resiliently flexible and allow the tip portion 54 to flex outwardly during the cutting of the bone so as to retrieve as large a sample of the bone as possible. Once the bone sample or core is secured in the central opening 22 of the biopsy stylet 50, the stylet is pulled upwardly through and out of the central opening 18 of the outer cannula 16.

During the procedure, the medical professional may now wish to retrieve a sample of tissue, such as bone marrow, of the patient. When positioned within the patient, one or more of the apertures 42, and preferably all of the apertures, of the outer cannula are disposed within and exposed to the bone marrow. The apertures 42, when exposed to the bone marrow, ensures that the bone marrow can be retrieved through multiple different apertures at different locations, thus preventing or minimizing the occurrence of hemodilution. The bone marrow can be retrieved directly through the apertures 42 of the outer cannula 16 by coupling the vacuum source 28 to the central opening 18. However, the bone marrow retrieved in this manner may cause undue or unnecessary pain to the patient because of the negative pressure created in the bone marrow during aspiration and/or may cause hemodilution of the bone marrow sample. As such, the medical professional can opt to employ the aspiration stylet 70 of the present invention to assist with the aspiration of the bone marrow. The biopsy stylet 50 can be removed from the outer cannula and the aspiration stylet 70 can be inserted into the central opening 18 of the outer cannula 16. The aspiration stylet 70 can be axially and rotationally positioned within the central opening 18 such that one or more apertures of the aspiration stylet 70 can be aligned with or disposed in registration with one or more apertures of the outer cannula 16. The aspiration stylet 70 can include a pair of internal lumens 74A, 74B, such that the fluid source 26 can be coupled to the open ended lumen 74A and the vacuum source 28 can be coupled to the closed end lumen 74B. The lumen 74A introduces the fluid to the surgical site during the bone marrow removal procedure so as to reduce or eliminate the pain experienced by the patient by minimizing the pressure differential in the bone marrow during removal. Specifically, as the vacuum source aspirates the bone marrow through the aligned apertures 42, 76 and into the opening 74B of the aspiration stylet 70, the fluid, such as saline, is introduced to the surgical site prior to and/or during tissue removal so as to reduce or minimize the pressure differential in the bone marrow at the surgical site. The present invention can reduce the pressure differential at the surgical site by greater than 80%, preferably greater than 90%, and most preferably greater than 97%, based on experimental results. The introduction of the fluid, such as saline, into the surgical site coupled with the aspiration of the tissue through multiple different apertures, at different locations, decreases the hemodilution of the resultant sample. According to one practice, the hemodilution can be reduced by greater than 40% and preferably greater than 50%, according to experimental results. The bone marrow can be removed from the patient at one or more selected axial locations and through different multiple apertures during the procedure. Specifically, the medical professional can retrieve a selected volume of bone marrow through each exposed aperture so as to minimize or avoid hemodilution. Further, the sealing element 80 formed on the outer surface 72A of the aspiration stylet 70 can form a fluid tight seal between the outer surface 72A of the aspiration stylet 70 and the inner surface 40B of the outer cannula 16. The seal 80 thus helps prevent fluid from entering the space or volume between the surfaces so as to further minimize the pressure differential within the bone marrow.

The apertures 42 formed in the outer cannula 16 can be arranged in any selected manner and can include for example a repeating pattern or some other type of aperture arrangement so that the location of an aperture can be easily correlated with an aperture in, for example, the aspiration style. The handle 24 can be used to manipulate the biopsy stylet 50 and the aspiration stylet 70 during use. The correlation between apertures can be used to open or close an aperture in the outer cannula 16, which in turn determines the marrow sampling location and area available to the aspiration stylet 70.

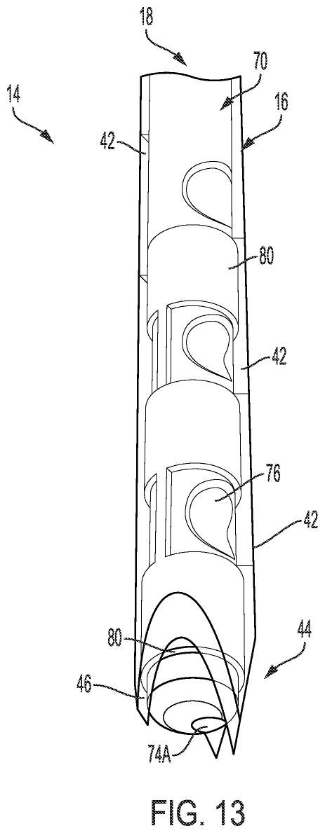

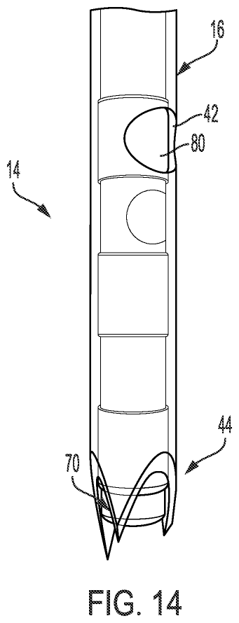

Further, the aspiration stylet 70 is movable relative to the outer cannula 16 so as to access different locations within the bone marrow, rather than moving the outer cannula 16 relative to the patient as is performed in the prior art. The ability to access one or more apertures in the outer cannula 16 by way of a movable aspiration stylet 70 is effectuated by aligning one or more of the apertures 42 of the outer cannula 16 with one or more apertures of the aspiration stylet 70. The occlusion of apertures 42 in the outer cannula 16 that are not aligned with a sampling aperture 76 occurs by default as the apertures 76 face the outer wall surface 72A or one or more sealing elements 80 of the aspiration stylet 70, as shown for example in FIGS. 13 and 14. By occluding the non-sampling apertures, the occluded apertures are in essence isolated from the one or more sampling apertures. This default occlusion of selected non-sampling apertures and the isolation of sampling apertures can be augmented by means of a sealing mechanism or element 80 disposed about or between the apertures.

According to one embodiment of the present invention, the tissue penetrating device 10 can include an outer cannula 16 and an inner cannula component 20 that each employs apertures (e.g., the outer cannula 16 and the aspiration stylet 70) where the apertures 42 of the outer cannula 16 can be optionally arranged at regular intervals along a spiral path for a given length of the main body. A similar spiral pattern of apertures can be employed on the aspiration stylet 70 as a mechanism for moving the stylet relative to the outer cannula. The handle 24 that is coupled to the inner cannula component 20 (e.g., the biopsy stylet 50 or the aspiration stylet 70) can turn or rotate the main body of the stylet within the outer cannula 16. The rotation, for example, of the aspiration stylet in the outer cannula 16 changes the position and location of the apertures 76 relative to the position and location of the apertures 42 of the outer cannula 16. By varying the rotation of the handle 24 and attached stylet relative to the main body 40 of the outer cannula 16, the depth and radial location of the sampling area within the bone can be varied without having to move the outer cannula 16 as in conventional devices. Thus, the outer cannula 16 can remain stationary during the procedure.

According to another embodiment, the apertures 42 of the outer cannula 16 can be formed along multiple different spiral paths which are of the same spiral parameters but at a different start point, similar to for example a multiple start screw thread or in opposing directions such as a double helix configuration. For example, the start point of the spiral path can be radially deviated by 90 degrees and therefore have four start positions. This would create numerous discrete areas of marrow sampling. Access to the apertures 42 can be varied in different embodiments with different configurations of apertures being formed in the components of the cannula assembly 14.

For example, in one embodiment, a single aperture 76 on the aspiration stylet 70 can be positioned via the handle 24 that sequentially aligns each aperture 76 individually with the apertures 42 formed in the outer cannula 16 that are positioned along a single spiral path. If there are multiple start points and multiple apertures at a single depth, the path traveled by the handle 24 and associated aspiration stylet 70 can have annular paths interspersed with spiral paths in the housing to allow sequential sampling of all apertures in the outer cannula 16.

Another embodiment of the present invention can include multiple apertures 76 formed in the aspiration stylet 70 that correspond to positions along each start of the spiral pattern of apertures 42 formed in the outer cannula 16. For example, the number of apertures 76 on the aspiration stylet 70 can equal the number of spiral starts for the aperture configuration of the outer cannula 16, such as for example four start points. The apertures 76 in the aspiration stylet 70 can be aligned radially with the spiral apertures on the outer cannula 16, such that all of the apertures in the aspiration stylet (e.g., four) align and open simultaneously to allow multi-depth or multi-positional sampling of bone marrow at a particular depth, or both, at four different positions simultaneously.

In still another embodiment, the present invention contemplates staggering the apertures 76 of the aspiration stylet 70 along the spiral path of each start point such that only a single aperture on a single start point is accessed at a time despite multiple apertures being formed in both the outer cannula and the aspiration stylet. This differing configuration of apertures is termed an interference pattern. Such a configuration can decrease the travel distance required in the handle to access the entire distance of the side apertures in the outer cannula by a factor of the number of starts, in this case, four.

The present invention also contemplates the use of an optional sealing element 80 for fluidly isolating the apertures 42 in the outer cannula 16 during the sampling or aspiration procedure, so as to improve the overall performance of the device 10 without relying on the small gap formed between the aspiration stylet and outer cannula, which typically provides a high resistance to flow. In the embodiment where the second lumen 74A in the aspiration stylet is provided so as to provide a flow of a fluid, such as saline, into the bone marrow at a distal end thereof so as to balance the negative pressure from aspiration, the saline may also inadvertently and unwantedly flow through the small gap between the inner and outer cannulas.

Different sealing element configurations and types of seals can be employed. For example, a ribbed seal or face seal (e.g., O ring, X-ring, bilobed face seal, etc.) over-molded onto the inner diameter (ID) of the outer cannula 16 or the outer diameter (OD) of the aspiration stylet 70 can function if there is only a single aperture at a given distance from distal tip of the needles. In the situation where there are multiple starts to the spiral pattern of the outer cannula, this embodiment may allow limited flow of fluid along the circumference of the aspiration stylet and an aperture at a different radial location can communicate with the intended aperture. In this case, a checkered pattern to the ribbed seal may suffice to seal flow in the perpendicular direction. However, the friction between the inner stylet and the outer cannula may be too great for adequate function. A standard face seal such as a quad-ring or O-ring may not suffice if there are multiple apertures at a given distance such as in multiple starts. The current sealing element can be positioned between the outer surface 72A of the aspiration stylet 70 and the inner surface 40B of the outer cannula 16. The sealing element can be conversely applied to the ID of the outer cannula 16.

If it is desirable to provide a second lumen in the inner cannula component 20, size constraints may require that the second lumen be formed by the seal itself rather than utilizing multiple stainless steel tubes and seals, and therefore the sealing may have to result from the interface of the ID of the outer cannula and a flexible tube extruded over a rigid lumen. In this case, a ribbed post-processing may improve seal performance and decreased resistance. These seal solutions are included in well-established prior art.

During a diagnostic bone sampling procedure, a trephine bone core, otherwise known as a bone marrow biopsy (BMB), is commonly obtained. There are two main issues with current or conventional devices and methods for BMB, including that the biopsy and aspiration cannot be done in the same bone puncture due to aspiration artifact, resulting in two punctures, and the core is frequently small or lost entirely (not retained in the capture device). The present inventors have developed a coaxial bone biopsy catching method and device that solves these issues.

By aspirating the bone marrow through a side aperture as described above allows a bone core biopsy and aspiration to be done through the same bone puncture. A biopsy stylet 50 can be inserted into the same outer cannula 16 as is used with the aspiration stylet 17. The core is taken from the distal end and the aspiration is done from the side apertures which eliminates or reduces the aspiration artifact.

Further, improvement in the core size and retention is accomplished by producing radial resistance between the core of bone and the biopsy stylet used to capture it. The resistance/force needs to overcome the intrinsic bone tissue tear/fracture force. Increased retention force is accomplished with accentuated compression of the bone core within the stylet used to capture it.

We claim:

1. A tissue penetrating device, comprising
an outer cannula having a main body having a tip portion formed at a distal end and a plurality of apertures formed at selected locations along an axial length thereof, the main body also forming a central opening that extends along the axial length,
a hub portion coupled to a proximal end of the outer cannula,
a biopsy stylet having a main body forming a central opening and having a tip portion formed at a distal end thereof having a beveled end portion, wherein the main body has a pair of opposed slits formed in the distal end and axially extending from the tip portion a selected length along the main body, wherein the slits formed in the biopsy stylet form at the distal end of the main body a pair of flexible opposed stylet halves, and
a handle portion coupled to a proximal end of the biopsy stylet,
wherein the biopsy stylet has an outer diameter smaller than an inner diameter of the outer cannula so as to seat within the central opening of the outer cannula and the handle has a selected portion that is sized and configured to seat within the hub portion.

2. The tissue penetrating device of claim 1, wherein the tip portion of the outer cannula comprises a plurality of prongs.

3. The tissue penetrating device of claim 1, wherein the biopsy stylet is movable relative to the outer cannula, and wherein the tip portion has a plurality of notches formed therein.

4. A tissue penetrating device, comprising
an outer cannula having a main body having a tip portion formed at a distal end and a plurality of apertures formed at selected locations along an axial length thereof, the main body also forming a central opening that extends along the axial length,
a hub portion coupled to a proximal end of the outer cannula,
an aspiration stylet having a main body having a tip portion formed at a distal end and a plurality of apertures formed at selected locations along an axial length thereof, the main body forming a central opening, and
a handle portion coupled to a proximal end of the aspiration stylet,
wherein the aspiration stylet has an outer diameter smaller than an inner diameter of the outer cannula so as to seat within the central opening of the outer cannula and the handle has a selected portion that is sized and configured to seat within the hub portion.

5. The tissue penetrating device of claim 4, wherein the tip portion of the outer cannula comprises a plurality of prongs.

6. The tissue penetrating device of claim 4, wherein the main body of the aspiration stylet has a central opening that includes a plurality of lumens that are fluidly isolated from each other.

7. The tissue penetrating device of claim 6, wherein a first lumen of the plurality of lumens has an open end at the tip portion of the main body of the aspiration stylet, and a second lumen of the plurality of lumens has a closed end at the tip portion.

8. The tissue penetrating device of claim 7, wherein the first lumen is configured and disposed to be in fluid communication with a fluid source for delivering a fluid through the open end of the tip portion, and wherein the second lumen is configured and disposed to be in fluid communication with a vacuum source for aspirating tissue through one or more of the apertures of the outer cannula and the aspiration stylet.

9. The tissue penetrating device of claim 4, wherein the plurality of apertures formed in the main body of the aspiration stylet are arranged such that when the main body of the aspiration stylet is moved one or more of the plurality of apertures is disposed in registration with one or more of the plurality of apertures of the outer cannula.

10. The tissue penetrating device of claim 6, wherein the aspiration stylet further comprises one or more sealing elements disposed on an outer surface of the main body.

11. The tissue penetrating device of claim 6, wherein the main body of the aspiration stylet includes an expansion portion for forming a seal between an outer surface of the aspiration stylet and an inner surface of the outer cannula.

12. A method for aspirating tissue, comprising
inserting an outer cannula within tissue of a patient at a selected location, wherein the outer cannula has a main body having a plurality of apertures formed at selected locations along an axial length thereof, the main body also forming a central opening that extends along the axial length thereof,
inserting a biopsy stylet within the central opening of the outer cannula and into the tissue of the patient so as to cut and retain a portion of the tissue, wherein the biopsy stylet is axially movable relative to the outer cannula,
removing the biopsy stylet from the central opening of the outer cannula to thus remove the portion of the tissue,
inserting an aspiration stylet into the central opening of the outer cannula and into the tissue of the patient, wherein the aspiration stylet has a main body having a plurality of apertures formed at selected locations along an axial length thereof, and
manipulating the aspiration stylet such that one or more of the apertures of the aspiration stylet align with one or more of the apertures of the outer cannula.

13. The method of claim 12, wherein the biopsy stylet is inserted into the central opening of the outer cannula, and further comprising
inserting together the outer cannula and the biopsy stylet into the tissue of the patient, wherein the tissue is cut during the insertion of the biopsy stylet.

14. The method of claim 12, wherein the aspiration stylet further includes one or more sealing elements mounted on an outer surface thereof, the method further comprising
positioning the aspiration stylet such that the one or more sealing elements seals one or more of the plurality of apertures of the outer cannula.

15. The method of claim 14, further comprising manipulating the aspiration stylet such that one or more of the plurality of apertures of the outer cannula are aligned with one or more of the plurality of apertures of the aspiration stylet in sequence starting from the distal most aperture of the outer cannula.

16. The method of claim 12, further comprising manipulating the aspiration stylet such that all of the plurality of apertures of the outer cannula are aligned with all of the plurality of apertures of the aspiration stylet.

17. A method for aspirating tissue, comprising
providing an outer cannula having a main body having a plurality of apertures formed at selected locations along an axial length thereof, the main body also forming a central opening that extends along the axial length thereof, the outer cannula being configured to be inserted into tissue of a patient,
providing an aspiration stylet sized and configured to be insertable within the central opening of the outer cannula and into the tissue of the patient, wherein the aspiration stylet has a main body having a plurality of apertures formed at selected locations along an axial length thereof, and
manipulating the aspiration stylet when inserted into the central opening such that one or more of the apertures of the aspiration stylet align with one or more of the apertures of the outer cannula.

18. A method for providing a biopsy and aspiration of tissue, comprising
providing an outer cannula having a main body having a plurality of apertures formed at selected locations along an axial length thereof, the main body also forming a central opening that extends along the axial length thereof, the outer cannula being configured to be inserted into tissue of a patient,
providing a biopsy stylet sized and configured to be insertable within the central opening of the outer cannula and into the tissue of the patient so as to cut and retain a portion of the tissue when inserted therein, wherein the biopsy stylet is axially movable relative to the outer cannula which can remain stationary,
providing an aspiration stylet sized and configured to be insertable within the central opening of the outer cannula and into the tissue of the patient, wherein the aspiration stylet has a main body having a plurality of apertures formed at selected locations along an axial length thereof, and
manipulating the aspiration stylet when inserted into the central opening such that one or more of the apertures of the aspiration stylet align with one or more of the apertures of the outer cannula.

19. A tissue penetrating kit, comprising
an outer cannula having a hub portion coupled to a proximal end and a tip portion formed at a distal end, the outer cannula having a main body having a plurality of apertures formed at selected locations along an axial length thereof, the main body also forming a central opening that extends along the axial length,
a biopsy stylet having a first handle portion coupled to a proximal end and a tip portion formed at a distal end, the biopsy stylet having a main body having a pair of opposed slits formed in the distal end and axially extending from the tip portion a selected length along the main body, wherein the tip portion has a beveled end portion and a plurality of notches formed therein, wherein the slits formed in the biopsy stylet form at the distal end of the main body a pair of flexible opposed stylet halves, and wherein the biopsy stylet has an outer diameter smaller than an inner diameter of the outer cannula, and
an aspiration stylet having a second handle portion coupled to a proximal end and a tip portion formed at a distal end, wherein an outer diameter of the aspiration stylet is smaller than an inner diameter of the outer cannula, and wherein the aspiration stylet has a main body having a tip portion formed at a distal end and a plurality of apertures formed at selected locations along an axial length thereof.
